(19) United States
(12) Patent Application Publication
(10) Pub. No.: US 2009/0297632 A1
(43) Pub. Date: Dec. 3, 2009

(54) Device, Methods and Compositions to Alter Light Interplay with Skin

(76) Inventor: Jacob M. Waugh, Mountain View, CA (US)

Correspondence Address: Jacob M. Waugh, #100, 2400 Bayshore Parkway, Mountain View, CA, 94043, US

(21) Appl. No.: 12/131,899

(22) Filed: Jun. 2, 2008

(51) Int. Cl. A61K 33/06

(52) U.S. Cl. 424/684

(57) ABSTRACT

Device, methods, and compositions to alter light interplay with skin to change appearance of wrinkles, pigmentation, stretch marks, and cellulite as well as other skin conditions are described. The present invention relates generally to the treatment of soft tissue volume deficiency and loss, facial wrinkles, facial lines, pigmentation patterns not desired by the subject, stretch marks, cellulite and more particularly, to treatments for volume restoration or coloration correction using light reflective materials such as micas and the like.

### FIELD OF THE INVENTION

This application relates to device, methods and compositions to alter light interplay with skin to change appearance of wrinkles, pigmentation, stretch marks, and cellulite as well as other skin conditions. The present invention relates generally to the treatment of soft tissue volume deficiency and loss, facial wrinkles, facial lines, pigmentation patterns not desired by the subject, stretch marks, cellulite and more particularly, to treatments for volume restoration or coloration correction using light reflective materials such as micas and the like.

### DESCRIPTION OF THE RELATED ART

Human skin is a composite material of the epidermis and the dermis. The top most part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by the structural changes in the skin due to aging and excessive sun exposure. The physiological changes associated with skin aging include impairment of the barrier function and decreased turnover of epidermal cells, for example (Cerimele, D. et al., Physiological Changes in Ageing Skin, British Journal of Dermatology, 122 Suppl. 35:13-20, April 1990).

The mechanical properties of the skin, such as elasticity, are controlled by the density and geometry of the network of collagen and elastic fiber tissue therein. Damaged collagen and elastin lose their contractile properties, resulting in skin wrinkling and skin surface roughness. As the skin ages or becomes unhealthy, it acquires sags, stretch marks, bumps, bruises or wrinkles, it roughens, and it has reduced ability to synthesize Vitamin D. Aged skin also becomes thinner and has a flattened dermoepidermal interface because of the alterations in collagen, elastin, and glycosaminoglycans (Fenske, N. A. and Lober, C. W., Structural and Functional Changes of Normal Aging Skin, Journal of American Academy of Dermatology, 15:571-585, October 1986; Montagna, W. and Carlisle, K., Structural Changes in Aging Human Skin, Journal of Investigative Dermatology, 73(1):47-53, 1979).

Augmentation of the skin can be an important factor in recovering from injury or for cosmetic purposes. For example, with normal aging, skin may become loose or creases can form, such as nasal-labial folds. In the face, creases or lines may adversely affect a person's self esteem or even a career. Thus, there has been a need for compositions and methods that can diminish the appearance of creases or lines.

Further, there are situations in which loss of tissue can leave an indentation in the skin. For example, surgical removal of a dermal cyst, lipoatrophy or solid tumor can result in loss of tissue volume. In other cases, injuries, such as gunshot wounds, knife wounds or other excavating injures may leave an indentation in the skin. Regardless of the cause, it can be desirable to provide a dermal filler that can increase the volume of tissue to provide a smoother or more even appearance.

Several compositions are available for such purposes. Collagen is often used as an injectable material for soft tissue augmentation. Additionally, numerous other materials, including proteins, fats, hyaluronic acid (HA), polyalcohols, and other polymers have been used as injectable dermal fillers. However, non-cross linked, hydrophilic polymers such as collagen, gelatin and HA have not performed well and must be covalently cross-linked to remain in place to be effective. One example is Zyderm®, which is uncrosslinked bovine collagen, was not effective as a dermal filler unless it was first cross-linked with glutaraldehyde to convert it to Zyplast®. Similarly, HA has not been sufficiently effective as a space filling material when injected or implanted in the body unless it is first cross-linked.

Proteins used for dermal augmentation, such as collagen, also must be cross-linked to perform well as dermal fillers. For example, Zyplast is a cross-linked bovine collagen dermal filler.

Numerous substances have been tested over the years for augmenting soft tissue in the dermis in the face to improve cosmesis by filling depressions in the skin (Klein, A. W. and Elson, M. L., The History of Substances for Soft Tissue Augmentation, Dermatological Surgery 26:1096-1105, 2000). This is an area that continues to be studied as there is no clearly superior material or product (Hotta, T., Dermal Fillers: The Next Generation, Plastic Surgical Nursing 24(1):14-19, 2004). These fillers are prepared from several polymers including bovine collagen, porcine collagen, chicken or bacteria fermented HA, gelatin, all of which are cross-linked covalently to reduce their dissolution time or immunological reactions. Fillers also include autologous human collagen (cross-linked collagen from the patient), and human cadaver dermis (cross-linked human collagen). Additional fillers are those that are insoluble in the dermis, including PMMA beads, dPTFE (expanded polytetrofluoroethylene), poly lactic acid, recombinant elastin, and thermoplastics that form gels when injected into humans (Klein and Elson). More recently, ceramic particles (U.S. Pat. No. 5,922,025) and also PMMA microspheres (Lemperle, G. et al, Migration Studies and Histology of Injectable Microspheres of Different Sizes in Mice, Plastic Reconstructive Surgery 113(5):1380-1390, 2004) have been used for soft tissue augmentation.

Dermal fillers are used to fill scars, depressions and wrinkles. Dermal filler substances have various responses in the dermis from phagocytosis to foreign body reactions depending on the material (Lemperle, G. et al., Human Histology and Persistence of Various Injectable Filler Substances for Soft Tissue Augmentation, Aesthetic Plastic Surgery 27(5):354-366; discussion 367, 2003). One goal of dermal fillers it to temporarily augment the dermis to correct the surface contour of the skin without producing an unacceptable inflammatory reaction, hypersensitivity reaction or foreign body reaction that causes pain, redness or excessive scar formation for a period of time.

One of the first materials to be used for dermal augmentation is Zyplast derived form bovine collagen. A newer material used for this application is Restylane® derived from bacteria-produced HA. Because challenges include both biocompatibility and persistence in the skin, new dermal fillers are compared to one of the existing products such as Zyplast or Restylane. (Narins et al., A Randomized, Double-Blind, Multicenter Comparison of the Efficacy and Tolerability of Restylane Versus Zyplast for the Correction of Nasolabial Folds, Dermatologic Surgery 29:588-595, 2003). However, there is a need for better compositions for use as dermal fillers.

The popular injectable fillers are minimally invasive to invasive cosmetic procedures used to treat the sign of aging. There is a variety of injectable fillers currently available on the market from collagen to HA to synthetic materials to restore more youthful appearance to aging skin. Injectable fillers can help fill in these lines and creases to provide skin with a plumper and smoother appearance. Most common injectable fillers are temporary and require repeated injections over time at a high price. For example, collagen injectable fillers are broken down and lose form while HA injectable fillers such as Restylane and Hylaform® are absorbed by the body. Reportedly permanent injectable fillers such as Artecoll® have a high risk of allergic reactions, side effects such as inflammatory nodules and bacterial infection. Furthermore, it is painful with side effect including swelling, bruising, bleeding and infection. With a rising demand for safer and longer lasting injectable filler, there is an increasing need for permanent filler with the benefits of HA and without adverse reactions of synthetic injectables.

Cellulite describes dimpled appearance of skin caused by deposit of fat. It is most common on thighs, hips and buttocks of females. While it is a harmless and normal condition, it is a well-known aesthetic problem and women spend fortunes on countless products and therapies to find a cure. A vast array of treatments such as topical formulation, dietary supplement, mesotherapy, laser, liposuction, massage or suction machines, body wraps, diet and exercise are available but there is no immediate effective treatment to eliminate cellulite. Some combination treatment may reduce the appearance of cellulite such as Velasmooth®, Endermologie® or TriActive®, but these options provide short-term benefits while requiring series of visit, time, maintenance and high costs. In addition, there are some side effects including redness, swelling, bruising and darkening of spider veins. Liposuction is surgical procedure to remove fat but this option may not diminish cellulite while being invasive, painful, expensive and including a recovery time. Diet and exercise is recommended to reduce appearance of cellulite with combination therapy. No single treatment has been proven to eliminate cellulite and combination therapy shows little benefits. As a result, an effective, safe, immediate solution to cellulite is needed.

Hyperpigmentation is a common skin discoloration where skin is darker than its surrounding skin. Skin contains cells called melanocytes that produce a brown skin coloring pigment melanin. An increase melanin causes hyperpigmented areas, brown spots or patches, as a result of sun damage, acne, or other skin disorders. Although, hyperpigmentation is difficult to treat and can be a long process, it can be treated by chemical peels, microdermabrasion, laser treatments or topical bleaching agents. Hydroquinone is the most commonly used topical agent, however, this product is irritating and cause allergic contact dermatitis and erythema. Treatments such as microdermabrasion or chemical peels require series of treatments and there is a risk of scarring and infection and not recommended for all individuals. Laser treatments are expensive and minor complications such as blistering, slight bleeding or burns as well as changes in pigment can occur. In most treatments, post inflammatory hyperpigmentation may occur. Since hyperpigmentation is difficult to treat with possible risks and has not been effectively managed for a wide population using currently available therapy, an innovative therapy is needed.

BOTOX® is a Botulinum Toxin Type A product manufactured by Allergan, Inc. It is a therapeutic agent that works at motor nerve endings to block the release of acetylcholine to paralyze the muscles and prevent them from contracting. Although originally slated to treat neurological disorders, BOTOX injections are widely used today to remove facial lines, especially frown lines, crow's feet and forehead lines. BOTOX injections are temporary and require repeated injections to maintain the results every 4-6 months with high costs for each treatment. The painful injection has many side effects which include bruising, headaches, respiratory infection, drooping of eyelids, nausea, flu syndromes, erythema, and muscle weakness. A noninvasive or less invasive alternate treatment is needed or botulinum toxin must be employed in conjunction with mechanical solutions such as fillers which require injection as well.

### SUMMARY OF THE INVENTION

In accordance with the present invention there is provided a method for augmenting volume of a soft tissue. This method involves the delivery of light reflective materials such as but not limited to mica, crystalline materials, metallic materials, as well as any light reflective material or material which under local skin or tissue conditions takes on the property of reflecting light back to some degree. The delivery step may be performed by syringe injection technique, tattoo gun, powder injection, pressure injection, patch, or topical with transdermal penetration. The soft tissue is selected from the group consisting of subdermal space, subcutaneous space, submucosal space, epidermal space deep to stratum corneum, immediate subdermal space and any combination of these structures or adjacent spaces or structures.

In accordance with one aspect of the present invention there is provided a method of treating subcutaneous volume deficiency in, for example, the face, lips, brow, peri-eyes, cheeks, buttocks, thighs, abdomen, breasts, and arm areas comprising injection or otherwise introducing past the stratum corneum of skin into the area being treated, such as via syringe injection or a tattoo gun.

In accordance with another aspect of the present invention there is provided a method of treating cellulite, undesired pigmentation, or stretch marks comprising injection or otherwise introducing a light reflective material past the stratum corneum of skin into the area being treated, such as via syringe injection or a tattoo gun. The invention may also be used to treat or ameliorate other skin conditions as will be apparent to one skilled in the art.

In accordance with other aspects of the present invention one or more therapeutic substances may be added to the material prior to the implantation thereof. The therapeutic substances may include one or more of the following: botulinum toxin, retinoids, other injectable filler materials such as hyaluronic acids, collagens and the like, growth factors, differentiation factors, hydrogels, polymers, antibiotics, anti-inflammatory medications, or immunosuppressive medications. The material may be injected into the area being treated through a needle and syringe, small cannula, tattoo gun apparatus or the like, or powder injection device. The material may be percutaneously or transmucosally injected into the area being treated. In accordance with another aspect of the present invention there is provided a device for injection of a soft light reflective tissue filler material.

There has thus been outlined rather broadly, the more important features of the invention in order that the detailed description hereof may be better understood. There are additional features of the invention that will be described hereinafter.

In this respect, before explaining at least one embodiment of the invention in detail, it is to be understood that the invention is not limited in its application to the details of construction and to the arrangements of the components set forth in the following description. The invention is capable of other embodiments and of being practiced and carried out in various ways. Also, it is to be understood that the phraseology and terminology herein are for the purpose of the description and should not be regarded as limiting.

Other objects and advantages of the present invention will become obvious to the reader and it is intended that these objects and advantages are within the scope of the present invention.

### DETAILED DESCRIPTION OF THE INVENTION

The following description relates to systems and methods for light reflective materials as a filler material for augmenting tissue volume or altering pigmentation level or patterns. As used herein, the term “augmenting” includes both restoring abnormal contours to a more normal state for cosmetic or reconstructive reasons, and adding to the volume of an existing soft tissue space for cosmetic or reconstructive reasons. As described herein, volume enhancement includes cosmetic intradermal, intraepidermal, subcutaneous or intramuscular volume enhancement of the face, lips, brow, peri-eyes, cheeks, buttocks, thighs, abdomen, breasts, and arm areas, volume enhancement for the treatment of age related and pathologic soft tissue atrophy, cosmetic volume enhancement of the breast or buttocks or alteration of pigmentation patterns or level.

The materials most useful for the present invention consist of reflective materials which allow some mechanical effects but must exert a light effect. These materials include metals, metal salts, mica and fine crystal materials as well as any light reflective material or material which under local skin or tissue conditions takes on the property of reflecting light back to some degree. These materials as will be apparent to those skilled in the art can contain coloring agents and any suitable vehicle for introduction. The base may be principally solid, liquid, gas, or transitional material such as suspension aerosol or the like. These materials can readily combine with other materials which contribute mechanical or biologic effects or some combination thereof. The method of introduction can be any that allows delivery past stratum corneum as will be apparent to those skilled in the art. The methods include but are not limited to injection, tattoo gun or the like, patches, needle arrays or micro-needle patches, powder introduction systems such as powder-ject or pressure systems, ionophoresis or electrical/magnetic field introduction, or transdermal delivery after topical administration.

This invention, in one use thereof, is directed to a method of treating an area with stretch marks including but not limited to those in the subcutaneous or deep dermal region of the face, buttocks, thighs, legs, hips, abdomen, lower back, breasts, and arm areas through injection or otherwise introducing a light reflective material past the stratum corneum of skin into the area being treated, such as via syringe injection or a tattoo gun. This aspect of the invention includes all wrinkles and signs of aging as well as corrective and aesthetically desired recontouring of structures. Selection of which light reflective material should be used can be based on a number of factors including depth and severity of the wrinkle or contour defect, volume to be introduced, skin texture in the areas to be treated, general patient skin color, skin thickness, age, elasticity, photodamage, any relevant systemic disease, degree of local laxity, degree of motion in local structures, color and intensity of pigmentation to be changed as well as desired outcome color and intensity where different from general patient skin tone.

This invention, in one use thereof, is directed to a method of treating an area which has undesirable contour due to underlying fat or connective tissue pattern in the face, lips, brow, peri-eyes, cheeks, buttocks, thighs, abdomen, breasts, and arm areas through injection or otherwise introducing a light reflective material past the stratum corneum of skin into the area being treated, such as via syringe injection or a tattoo gun. Particularly useful are treatments for cellulite patterns which can be both treated and aesthetically improved by introduction of a light reflective material preferentially in the dimpled areas. Selection of which light reflective material should be used can be based on a number of factors including depth and severity of the cellulite or contour defect, volume to be introduced, skin texture in the areas to be treated, general patient skin color, skin thickness, age, elasticity, photodamage, degree of motion in local structures, any relevant systemic disease, color and intensity of pigmentation to be changed as well as desired outcome color and intensity where different from general patient skin tone.

This invention, in one use thereof, is directed to a method of treating a volume-deficient area in the subcutaneous or deep dermal region of the face, lips, brow, peri-eyes, cheeks, buttocks, thighs, abdomen, breasts, and arm areas through injection or otherwise introducing a light reflective material past the stratum corneum of skin into the area being treated, such as via syringe injection or a tattoo gun. This aspect of the invention includes all wrinkles and signs of aging as well as corrective and aesthetically desired recontouring of structures. Selection of which light reflective material should be used can be based on a number of factors including depth and severity of the wrinkle or contour defect, volume to be introduced, skin texture in the areas to be treated, general patient skin color, skin thickness, age, elasticity, photodamage, any relevant systemic disease, degree of local laxity, degree of motion in local structures, color and intensity of pigmentation to be changed as well as desired outcome color and intensity where different from general patient skin tone.

This invention, in one use thereof, is directed to a method of treating an area which has undesirable pigmentation the face, lips, brow, peri-eyes, cheeks, buttocks, thighs, abdomen, breasts, and arm areas through injection or otherwise introducing a light reflective material past the stratum corneum of skin into the area being treated, such as via syringe injection or a tattoo gun. Tailoring which light reflective material should be used can be based on a number of factors including general patient skin color, skin thickness, age, elasticity, photodamage, any relevant systemic disease, degree of motion in local structures, color and intensity of pigmentation to be changed as well as desired outcome color and intensity where different from general patient skin tone. The types of pigmentation include but are not limited to melasma, freckles, sun damage, age spots, scars, and keratoses.

The invention may also be used to treat or ameliorate other skin conditions as will be apparent to one skilled in the art.

In accordance with other aspects of the present invention one or more therapeutic substances may be added to the material prior to the implantation thereof. The therapeutic substances may include one or more of the following: botulinum toxin, retinoids, other injectable filler materials such as hyaluronic acids, collagens and the like, growth factors, differentiation factors, hydrogels, polymers, antibiotics, anti-inflammatory medications, or immunosuppressive medications. The material may be injected into the area being treated through a needle and syringe, small cannula, tattoo gun apparatus or the like, or powder injection device. The material may be percutaneously or transmucosally injected into the area being treated.

In accordance with another aspect of the present invention there is provided a device for injection of a soft light reflective tissue filler material.

The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be resorted to, falling within the scope of the invention.

### EXAMPLES

Wrinkles

A 64 year old African-American male with no relevant medical history presents with deep, defined laugh lines around his mouth. He hopes to minimize the appearance of wrinkles, and thus undergoes a tattoo treatment with mica that is one to two grades lighter than his skin tone. The mica is carefully introduced as an emulsion into the hollow of each wrinkle, mimicking its exact shape and size. The tattoo neutralizes the appearance of laugh lines by brightening the depressions of wrinkles and providing a filler volume contour effect.

A 38 year old Caucasian female with no relevant medical history seeks treatment for distinct crow's feet markings in the sensitive skin around her eyes. The wrinkles branch out from the corners of her outer eye and a few wrinkles are present beneath her eye as well. She receives a tattoo of mica that is one to two grades lighter than her skin. The tattoo application precisely traces and shades in the wrinkles, with the deepest lines receiving mica that is two grades lighter than skin tone. As a result, the dark crow's feet lines fade—the depressions are masked by the brightness of the mica—and are much less detectable and the material provides a filler effect which spreads and open the wrinkles mechanically as well.

An 82 year old Caucasian female with no relevant medical history seeks treatment for distinct brow lines. She receives injections of metallic and crystalline light reflective material in a base of hyaluronic acid with type a botulinum toxin. The light reflective material is moderately lighter than her skin. The injection pattern follows the wrinkles, with the deepest lines receiving mica that is two grades lighter than skin tone and a larger volume of injection. As a result, brow lines improve dramatically through the combined filler, reflective, and botulinum toxin effects which also allow changes in durability of the benefits.

Discoloration

A 58 year old Latin-American female with no relevant medical history presents with advanced age spots as well as sun damage to her face. Together, these have culminated in variegated facial discolorations. She receives a tattoo treatment of mica varying from two grades to three grades lighter than her skin tone, and immediately notices an improvement in her appearance. The discolorations are significantly less defined and less discernible.

A 14 year old Asian woman complains that her fair complexion is peppered with a number of dark freckles, leaving her unsatisfied with her appearance. Scars on her legs do not appear to take on a darker appearance as part of the healing process so she is identified as a good candidate for a more extensive or facial treatment. Mica that is three grades lighter than her skin tone is tattooed onto the individual freckles. This treatment leaves her with freckles that are appreciably less noticeable, especially from a distance. The appearance of freckles has diminished dramatically, some spots to the point that they are no longer visible.

Stretch Marks

After her pregnancy, a woman notices unsightly stretch marks on her lower abdomen. The lines are markedly lighter than skin tone and branch out over a moderate surface area. To correct this, she undergoes a tattoo treatment of mica that is two grades darker than her skin tone. The tattoo application closely traces the borders of the stretch marks and shades them in. The darker mica reduces the appearance of stretch marks by neutralizing their lighter-than-skin-tone color and providing a contour effect. This patient had prior reflective material injections as well.

After a rapid weight gain, a teenage girl discovers pale stretch marks extending over her inner thighs. To get rid of the marks, she requests mica injections from her physician. Mica that is two grades darker than her skin tone is tattooed directly onto the stretch marks, covering them completely. Post-treatment, the stretch marks are markedly less visible and the filler effect masks or reverses much of the thinning previously present in the treated area.

Cellulite

Finding the cellulite in her buttocks and thighs unattractive, a young woman seeks to camouflage its appearance and texture. The cellulite, or irregularities in fat content underneath the skin, results in visible lumps and dimples. Her physician tattoos the indentations or darker areas in her skin with mica that is one grade lighter than her skin tone. While the treatment does not remove or reduce the underlying issues which lead to the cellulite, it dramatically improves appearance by counteracting the shadows that are caused by raised skin and the resulting depressions.

What is claimed is:

1. A method of treatment or prophylaxis of age-related changes in human tissue comprising at least one light reflecting material in a pharmacologically acceptable formulation, wherein the material is introduced deep to the outermost layers of the skin.

2. A method according to claim 1, wherein said administration is dermal administration.

3. A method according to claim 1, wherein said administration is a single administration.

4. A method according to claim 1, wherein a light reflecting material is mica.

5. A method according to claim 1, wherein treatment or prophylaxis is an improvement of wrinkles and lines.

6. A method according to claim 1, wherein treatment or prophylaxis is treating hyperpigmentation.

7. A method according to claim 1, wherein treatment or prophylaxis is augmentation of tissue.

8. A method according to claim 1, wherein treatment or prophylaxis is altering tissue properties.

9. A method of treatment or prophylaxis of aesthetic defeat in tissue of a human in need of such treatment or prophylaxis, the method comprising at least one light reflecting material in a pharmacologically acceptable formulation, wherein the material is introduced deep to the outermost layers of the skin.

10. A method according to claim 1, wherein said administration is dermal administration.

11. A method according to claim 1, wherein said administration is a single administration.

12. A method according to claim 11, wherein a light reflecting material is mica.

13. A method according to claim 11, wherein treatment or prophylaxis is treating hyperpigmentation.

14. A method according to claim 11, wherein treatment or prophylaxis is treating redness of skin.

15. A device to improve aesthetic appearance for a patient in need thereof wherein said device comprises light reflective material introduced deep to the outermost layers of the skin.

16. A method according to claim 15, wherein said administration is dermal administration.

17. A method according to claim 15, wherein said administration is a single administration.

18. A method according to claim 15, wherein a light reflecting material is mica.

19. A method according to claim 15, wherein treatment or prophylaxis is an improvement of wrinkles and lines.

20. A method according to claim 15, wherein treatment or prophylaxis is treating hyperpigmentation.

21. A method according to claim 15, wherein treatment or prophylaxis is treating redness of skin.

22. A method according to claim 15, wherein treatment or prophylaxis is augmentation of tissue.

23. A method according to claim 15, wherein treatment or prophylaxis is altering tissue properties.
